(19) United States
(12) Patent Application Publication
(10) Pub. No.: US 2023/0041785 A1
(43) Pub. Date: Feb. 9, 2023

(54) BIOPSY DEVICE AND METHOD FOR TISSUE SAMPLING IN MAMMALS

(71) Applicants: Niclas ROXHED, Bromma (SE); Mikael SANDELL, Årsta (SE); Göran STEMME, Lidingö (SE); Staffan HOLMIN, Bromma (SE); Stefan JONSSON, Sollentuna (SE); Wouter METSOLA VAN DER WIJNGAART, Sollentuna (SE)

(72) Inventors: Niclas ROXHED, Bromma (SE); Mikael SANDELL, Årsta (SE); Göran STEMME, Lidingö (SE); Staffan HOLMIN, Bromma (SE); Stefan JONSSON, Sollentuna (SE); Wouter METSOLA VAN DER WIJNGAART, Sollentuna (SE)

(21) Appl. No.: 17/790,621

(22) PCT Filed: Jan. 4, 2021
(86) PCT No.: PCT/SE2021/050003

§ 371 Date: Jul. 1, 2022

(30) Foreign Application Priority Data
Jan. 3, 2020 (SE) 2030002-6

(51) Int. Cl. A61B 10/02; A61B 10/04

(52) U.S. Cl. CPC A61B 10/0233; A61B 10/04; A61B 2010/045

(57) ABSTRACT

A microbiopsy device for extracting a tissue sample, the microbiopsy device comprising a main body extending between a distal end and a proximal end and adapted to have or assume a shape of substantially uniform transverse width along the length of the main body, wherein the distal end is arranged to enter tissue; the proximal end comprises a mounting interface adapted for connection with an elongated member, or is integrally formed with a solid elongated member; the transverse width of the main body is smaller than 1 mm; and the main body comprises a recess extending in a longitudinal direction of the main body and defining a cavity arranged to capture tissue therein.

### TECHNICAL FIELD

The present disclosure generally relates to a micro biopsy device and a biopsy method to sample tissue. The biopsy method involves introducing and maneuvering the device via the vasculature into the tissue of a subject and obtaining a biopsy sample

### BACKGROUND ART

Biopsies are the gold standard in disease diagnostics, whether it relates to cancer, tissue rejection or otherwise unknown lesions. Several modalities exist for obtaining biopsies, including, but not limited to, fine needle biopsies, endoscopy, bronchoscopy or endovascular catheters. Endovascular interventional devices are often sub-millimeter in size and are used for a wide range of applications, however, none of those include taking biopsies from organs other than the heart. Utilizing other modalities for certain organs imposes other risks, due to the vicinity of other sensitive parts of the body.

WO 2020/089422 A1 discloses a biopsy device comprising a tip member with a rearward facing cutting portion for use in a biopsy procedure. The tip member is located towards the distal end of the elongate rod member and positioned so that the longitudinal axis of the tip member is angularly offset with respect to the longitudinal axis of the elongate rod member, wherein the distal end of the tip member is configured to penetrate tissue of a subject and the proximal end of the tip member comprises a cutting portion for cutting tissue of a subject. However, due to the angular offset between the longitudinal axes of the tip member and the rod member, the distal tip member is wider than the proximal rod member which limits navigability of the biopsy device in endovascular procedures.

US 2008/0167576 AA and US 2008/0208076 AA disclose a micro spike having a three-dimensional structure made of single crystalline silicon and being capable of taking a sufficient amount of a tissue to examine while minimizing an examinee's pain with a minimal invasion. The micro spike comprises a main body having several prongs extending from one side surface of the main body, and barbs extending from side surfaces of the prongs. The main body has a width of 100 μm to 50 mm, preferably 1 mm to 5 mm, and is attached to the end part of a wire in an endoscope for use in biopsy procedures.

WO 2013/155557 A1 discloses a microbiopsy device for taking biological samples, comprising a body including two or more cutting elements for cutting tissue to form a biological sample; and a chamber inside the body for receiving and retaining the biological sample, the chamber having an opening between the cutting elements, wherein the cutting elements are arranged to cut a section of tissue having a width of less than 1 mm.

US 2011/0071428 AA discloses a biopsy device which can be used with a flexible endoscope in minimally invasive procedures on the gastrointestinal system and other organs. Actuation devices for use to twist or manipulate the biopsy device even when it is at the end of the endoscopic cable are provided.

However, the previously mentioned biopsy devices exhibit a proximal portion that is considerably wider than the distal tissue capturing portion, in order to provide an interface for connection to endoscopes. As a result, the known devices do not reduce the size of already existing biopsy catheters, which limits which areas of the body can be reached.

Thus, there is a need to improve the known biopsy devices and methods to overcome the disadvantages mentioned above.

### SUMMARY OF INVENTION

An object of the present invention is therefore to achieve a minimally invasive biopsy device and method which reduces tissue damage at the site of sampling and enables extracting tissue samples from areas of the body which are not reachable with conventional devices.

This objected is achieved in a first aspect of the present disclosure in which there is provided a microbiopsy device for extracting a tissue sample, the microbiopsy device comprising a main body extending between a distal end and a proximal end and adapted to have or assume a shape of substantially uniform transverse width along the length of the main body, wherein the distal end is arranged to enter tissue; the proximal end comprises a mounting interface to enable connection with an elongated member, or is integrally formed with a solid elongated member; the transverse width of the main body is smaller than 1 mm; and the main body comprises a recess extending in a longitudinal direction of the main body and defining a cavity arranged to capture tissue therein.

By providing a microbiopsy device of a substantially uniform width or thickness smaller than 1 mm along its length, the present disclosure achieves a biopsy device which may introduced and advanced through the vasculature of a subject to reach sampling sites in the body not attainable with known devices. At the same time, the size of the tissue sample extracted in relation to the size of the microbiopsy device is maximized to provide a minimally invasive device with sufficient sampling for subsequent analysis.

The microbiopsy device may adapted to be attached to the distal end of an elongated member, e.g. a wire or tube, via the mounting interface provided on the proximal end of the main body. Alternatively, the microbiopsy device may be formed by machining the recess at the distal end of a solid elongated member, e.g. a wire, in which case the proximal end of the main body will be integrally formed with the solid elongated member.

In one embodiment, the transverse width of the main body is smaller than 500 μm, preferably smaller than 200 μm, most preferably smaller than 150 μm. The reduced width of the main body achieves a microbiopsy device which is an order of magnitude smaller than commercially available biopsy catheters. The present disclosure provides a microbiopsy device having a width comparable to that of a human hair.

In one embodiment, the volume of the cavity is smaller than 1 preferably smaller than 250 nl, most preferably smaller than 100 nl. The reduced volume of the cavity enables a small, minimally invasive microbiopsy device whilst capturing sufficient sample volumes of subsequent analysis.

In one embodiment, the recess comprises an opening in a transverse direction of the main body. The transverse opening facilitates lateral tissue capture when the microbiopsy device is introduced into tissue at the sampling site.

In one embodiment, the recess extends towards the distal end of the main body, terminating in a distal opening. Preferably, the recess extends along substantially the whole length of the main body. In this way, the size of the tissue capturing cavity is increased without increasing the overall size of the microbiopsy device.

In one embodiment, the main body has a substantially cylindrical shape. The cylindrical shape ensures that the microbiopsy device conforms to a circular cross-section of an endoluminal access device such as e.g. a catheter, sheath etc. used for introducing and advancing the microbiopsy device to the sampling site through the vasculature of the subject. Additionally, the cylindrical shape maximizes the volume of the microbiopsy device in relation to the transverse width.

In one embodiment, the microbiopsy device further comprises a plurality of barbs arranged within the recess and adapted to retain captured tissue. The barbs facilitate tissue capturing and ensure that the tissue sample remains intact in the cavity during extraction. The number of barbs is, for example, in the range 0-100, preferably in the range 2-50, advantageously in the range 4-25.

In one embodiment, the recess comprises a through-going opening to form two or more opposing prongs. Preferably, the prongs are flexible and adapted to be brought between a closed position, wherein the prongs are oriented in a longitudinal direction of the main body, and an open position, wherein the prongs expand beyond the transverse width of the main body. The flexible prongs may thus open laterally to a width greater than the main body to increase the size of the tissue sample captured.

In one embodiment, the prongs comprise a bumper structure arranged on an outer surface. The microbiopsy device comprising prongs may be used in conjunction with a tube that facilitates the opening and closing mechanism, wherein this mechanism facilitates the gripping and catching of tissue. The tube may be smaller than 1.2 mm in outer diameter, and smaller than 1 mm in inner diameter.

In one embodiment, the prongs have a thickness smaller than 500 μm.

In one embodiment, the microbiopsy device is manufactured in a material selected from a ceramic, a semiconductor, a metal or a polymer, or a combination thereof. Preferably in silicon or a polymer with a young's modulus larger than 1 GPa.

In one embodiment, the microbiopsy device is manufactured through additive manufacturing or using silicon micromachining technologies.

In a second aspect of the present disclosure, there is provided a biopsy device comprising: an endoluminal access device having a flexible elongated hollow body terminating in a distal penetration portion arranged to penetrate a vascular tissue wall, wherein an outer diameter of the endoluminal access device is smaller than 1.2 mm; an elongated member movably arranged inside the endoluminal access device and having an outer diameter corresponding to an inner diameter of the endoluminal access device; and a microbiopsy device according to the first aspect mounted on or integrally formed with a distal end of the elongated member and having a transverse width substantially equal to the outer diameter of the elongated member.

By means of the biopsy device, navigation using the vasculature of the subject as a pathway to reach the sampling site is achieved. The microbiopsy device, mounted on or integrally formed with the elongated member, is maintained in a retracted position inside the lumen of the endoluminal access device as the latter is advanced through the vasculature to the vicinity of e.g. an organ of interest. The distal sharp end is then used to penetrate the blood vessel wall, and subsequently the microbiopsy biopsy may be introduced into the organ by pushing the elongated member in a distal direction to capture a tissue sample in the cavity.

The biopsy device according to the preceding claim, wherein the elongated member is made from a shape-memory alloy, preferably nitinol.

In a third aspect of the present disclosure, there is provided a method of extracting a tissue sample from a subject comprising:

providing a biopsy device according to the second aspect;

introducing the biopsy device into the vasculature of the subject with the microbiopsy device and elongated member in a retracted position wherein the distal end of the main body does not protrude from the distal penetration portion of the endoluminal access device;

advancing the biopsy device through the vasculature until a targeted sampling site is reached;

penetrating the vascular tissue wall using the distal penetration portion of the endoluminal access device;

advancing the elongated member through the endoluminal access device such that the microbiopsy device exits from the distal penetration portion of the endoluminal access device and enters into tissue at the sampling site to capture a tissue sample;

retracting the elongated member into the endoluminal access device such that the microbiopsy device re-enters the distal penetration portion of the endoluminal access device with the captured tissue sample; and retracting the biopsy device from the vasculature.

In one embodiment, the step of advancing the biopsy device through the vasculature comprises navigating inside the vasculature using x-ray guidance.

A method of obtaining a tissue sample providing a tissue sampling device of less than 1 mm in width and 3 mm in length, wherein the device is inserted into tissue and, when retracted, collects the tissue.

### BRIEF DESCRIPTION OF DRAWINGS

The invention is now described, by way of example, with reference to the accompanying drawings, in which:

FIGS. 1a and 1b show perspective and cross-sectional views of one embodiment of the present disclosure.

FIGS. 2a and 2b show perspective and cross-sectional views of one embodiment of the present disclosure.

FIGS. 3a and 3b show perspective and cross-sectional views of one embodiment of the present disclosure.

FIGS. 4a and 4b show perspective and cross-sectional views of one embodiment of the present disclosure.

FIGS. 5a to 5k show side views of different arrangements of barbs used in conjunction with the embodiment of FIGS. 4a and 4b.

FIGS. 6a and 6b show perspective and cross-sectional views of one embodiment of the present disclosure.

FIG. 7 illustrates a method of taking a tissue sample by means of a microbiopsy device according to the embodiment of FIGS. 4a and 4b.

FIG. 8 shows a microscope view of one embodiment of the present disclosure.

FIGS. 9a to 9d show microscope views of the embodiment of FIGS. 4a and 4b during sampling.

### DESCRIPTION OF EMBODIMENTS

In the following, a detailed description of a microbiopsy device according to the present disclosure is presented. In the drawing figures, like reference numerals designate identical or corresponding elements throughout the several figures. It will be appreciated that these figures are for illustration only and are not in any way restricting the scope of the invention.

In the context of the present disclosure, it is understood that the terms “distal” and “distally” refer to a position or direction (furthest) away from the operator when using the microbiopsy device according to the present disclosure. Correspondingly, the terms “proximal” and “proximally” refer to a position or direction closest to or towards the operator when using the microbiopsy device according to the present disclosure.

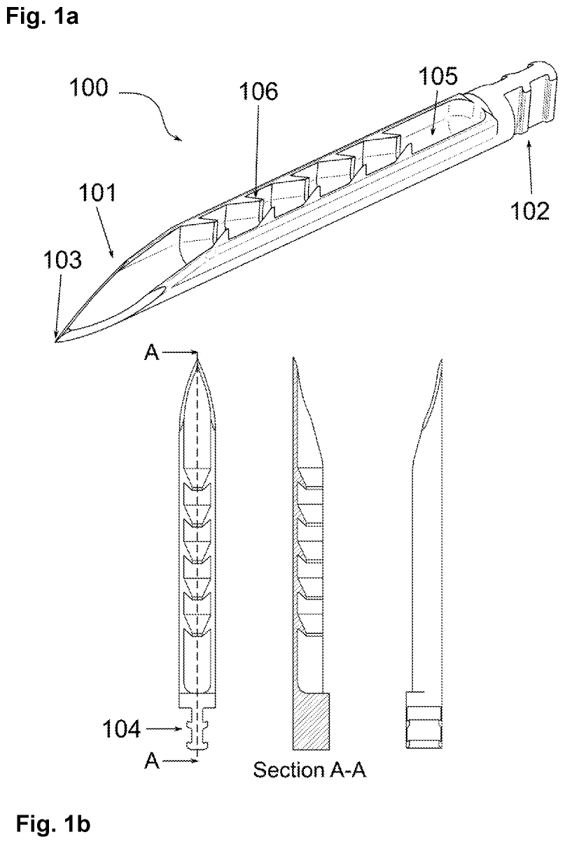

Referring to FIG. 1, there is shown one embodiment of a microbiopsy device according to the present disclosure. The microbiopsy device comprises a main body 100 extending between a distal end 101 and a proximal end 102. The distal end is arranged to enter tissue, for instance by means of a sharp beveled distal tip 103 as shown in FIG. 1. Said sharp distal tip preferably has an edge radius less than 50 μm, more preferably less than 5 μm. However, the distal end need not be sharp in order to enter tissue. By virtue of having a higher stiffness than the typically soft tissue intended to be sampled, the microbiopsy device according to the present disclosure may enter the tissue without requiring a sharp tip.

At the proximal end of the main body, there is provided a mounting interface 104 for connection to an elongated member to form a biopsy device which may be introduced and guided through the vasculature, as will be further explained below. In the embodiment in FIG. 1, the proximal end is machined to form part of an interlocking mechanism presenting two pairs of opposing lateral projections. Other shapes of the mounting interface are also contemplated.

The main body has a substantially uniform transverse width or thickness along the length of the main body. The transverse width is smaller than 1 mm, preferably smaller than 500 μm, more preferably smaller than 200 μm, most preferably smaller than 150 μm. In one embodiment, the transverse width is 122 μm, comparable to a human hair. The small width enables the microbiopsy device to be guided through the smallest passages of the vasculature to reach sampling sites unattainable by conventional biopsy devices whilst reducing tissue damage. The length of the main body is smaller than 10 mm, such as smaller than 7 mm, smaller than 5 mm, smaller than 3 mm. In one embodiment, the ratio between the length and width of the main body is from 10:1 to 2:1.

For capturing tissue at the sampling site, the main body comprises a recess 105 extending in a longitudinal direction of the main body, the recess defining a cavity which is arranged to capture and retain tissue therein. As may be seen in FIG. 1, the recess extends and opens towards the distal end of the main body, substantially the whole length of the main body. Of course, other examples of extension length are encompassed in the present disclosure. The volume of the cavity is smaller than 1 μl, preferably smaller than 250 nl, most preferably smaller than 100 nl. The combination of a microbiopsy device with a substantially uniform transverse width with a longitudinal recess optimizes the size of the tissue sample that can be extracted in relation to the size of the device, to ensure a minimally invasive microbiopsy capable of extracting sufficient tissue to allow for subsequent analysis.

The recess in the embodiment shown in FIG. 1 is open in a transverse direction of the main body to facilitate capture of tissue in a lateral direction. However, the recess may also only be open toward the distal end, as will be shown in other embodiments.

The main body has a substantially cylindrical shape to conform to a circular lumen of an endoluminal access device, such as a sheath or catheter. Other cross-sectional shapes are also contemplated within the present disclosure.

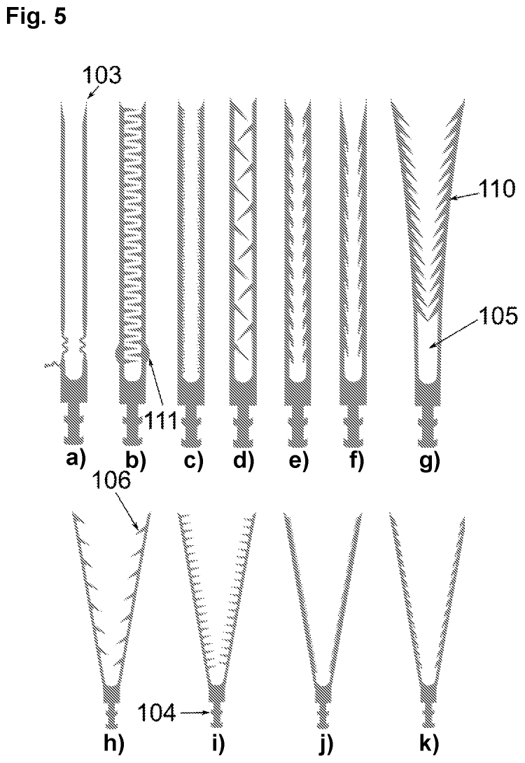

Within the recess, there is provided a number of barbs 106 in the form of proximally oriented projections which serve to grip, secure and retain the sampled tissue in the cavity. The barbs may also assist in severing the tissue at the sampling site. In the embodiment of FIGS. 1a and 1b, the recess comprises five barbs which extend in a circumferential direction along the inner walls of the cavity. Other configurations and numbers of barbs are also encompassed in the present disclosure as shown in FIGS. 5a to 5k. The number of barbs may be between 0 and 100, such as in the range 2-50, such as in the range 4-25. The barbs may be arranged directly opposite one another, or in an alternating configuration along the length of the recess.

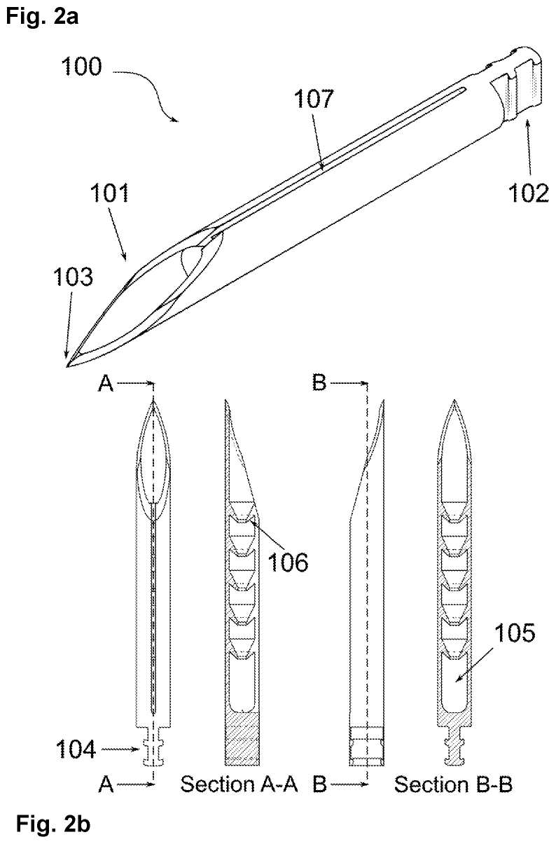

Referring now to FIGS. 2a and 2b, there is shown another embodiment of a microbiopsy device according to the present disclosure. This embodiment is similar to the one in FIG. 1, but the recess is nearly closed in the transverse direction, except for a longitudinal slit 107 extending along the length of the main body.

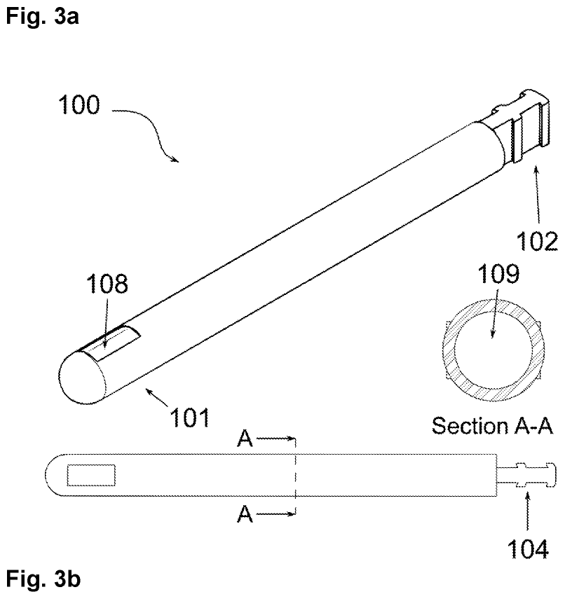

Referring now to FIGS. 3a and 3b, there is shown another embodiment of a microbiopsy device according to the present disclosure. In this embodiment, the distal end is rounded and does not exhibit a sharp distal tip. Furthermore, the main body is substantially tubular with only a transverse opening 108 near the distal end from the internal cavity 109 of the recess. The opening may be covered by a membrane which is arranged to rupture as the microbiopsy device is introduced into tissue. A vacuum or negative pressure may be provided in the cavity which then serves to draw tissue into the recess when the membrane is ruptured. In an alternative embodiment (not shown) the opening is provided in the distal end, oriented coaxially with the main body.

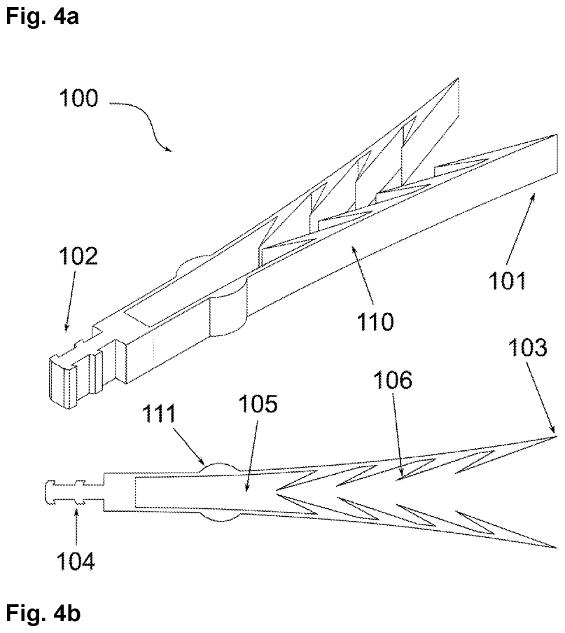

Referring now to FIGS. 4a and 4b, there is shown another embodiment of a microbiopsy device according to the present disclosure. This embodiment comprises two angled prongs 110, each with a sharp cutting edge near the distal end for low penetration force, bumper structures 111 on the outside of the prongs to enable the gripping action and backwards-facing sharp barbs for tearing of tissue during retraction.

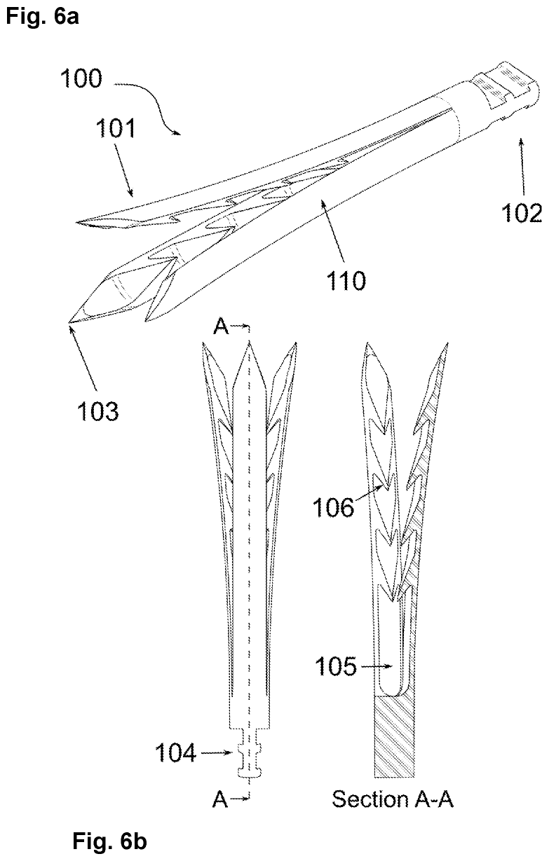

Referring now to FIGS. 6a and 6b, there is shown another embodiment of a microbiopsy device according to the present disclosure, similar to the embodiment of FIGS. 4a and 4b. This embodiment comprises three angled prongs 110, each with a sharp cutting edge near the distal end for low penetration force, bumper structures 111 on the outside of the prongs to enable the gripping action and backwards-facing sharp barbs for tearing of tissue during retraction. The number of prongs may be four (not shown).

WO 2009/124990 A1 discloses an endoluminal medical access device, hereinafter called Extroducer, that is devised for endoluminal delivery to an extravascular target site at a vasculature site of a human or animal body vasculature, such as the microvasculature. The device comprises a hollow body arranged around a continuous channel that ends in a distal end and comprises a distal penetration portion that is devised to extend across a tissue wall of said microvasculature said microvasculature site at an extravascular target site in said body to provide communication with said extravascular target site through said channel and devised for at least partly apposition to said tissue wall, and a proximal connection section, which proximally adjoins said penetration portion, and optionally comprises an intrusion depth limit unit and/or a hollow separation section devised to provide a controllable separation of the penetration portion from a connected proximal portion of the hollow body. The contents of WO 2009/124990 A1 are incorporated herein by reference in its entirety.

The Extroducer is a trans-vessel wall catheter of 0.25 mm in outer diameter that can be navigated to almost anywhere in the body using x-ray guidance. Operating a microbiopsy device according to the present disclosure in combination with the Extroducer reduces the size of the biopsy system, whilst maximizing the size of the sample in relation to the damage inflicted at the sampling site. In one aspect, the microbiopsy device is mounted on Nitinol wires that can move inside the Extroducer system.

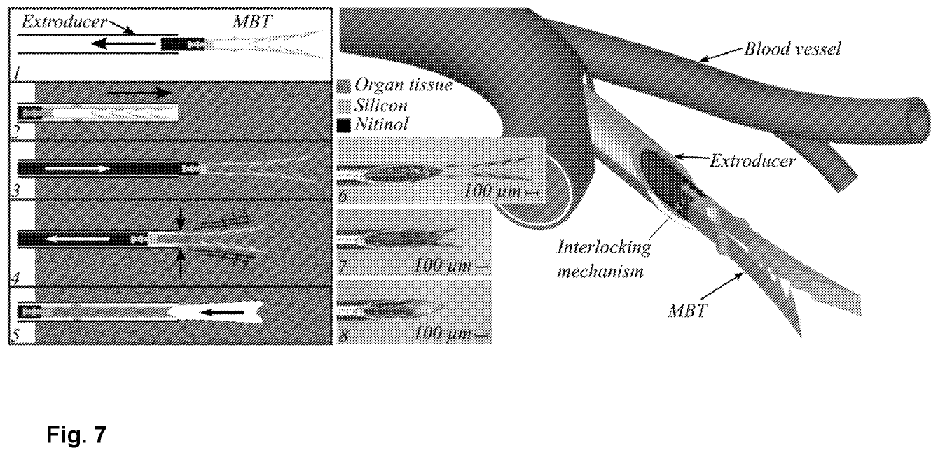

Referring now to FIG. 7, an overview of the operating principle of the microbiopsy device according to one embodiment of the present disclosure is illustrated. Loading of the microbiopsy device into the Extroducer (image 1), system insertion into tissue (image 2), microbiopsy device insertion and gripper opening (image 3), microbiopsy device retraction and gripper closing (image 4) and a resulting sample protected inside the Extroducer (image 5). Images 6-8 show a microscope image sequence corresponding to images 3-5 in air. Image 9 shows a CAD rendering of the intended usage in the vasculature. The Extroducer is designed to exit the blood vessel into surrounding tissue, after which the microbiopsy device is inserted into the tissue and retracted.

Loading the microbiopsy device into the Extroducer prior to operation closes the gripper through the mechanical interaction between the bumpers and the Extroducer (FIG. 7, images 1-2). The entire system is designed to use the vasculature as a pathway to reach the sampling site, aided by x-ray guidance, (FIG. 7, image 9). The microbiopsy device is protected inside the Extroducer while the Extroducer penetrates the blood vessel wall and enters the organ of interest. After correct positioning, the Extroducer is kept in place while the microbiopsy device is pushed axially into the organ. During this insertion, the gripper opens laterally due to the built-in stress. When subsequently retracting the gripper closes laterally due to the bumper structures pushing the prongs. Retraction of the microbiopsy device thus creates a gripping action that traps the sample and pulls it back into the Extroducer (FIG. 7, images 3-5). The microbiopsy device and sample are protected inside the Extroducer and the catheter is extracted.

We assumed typical microbiopsy device penetration forces in soft tissues of 10 mN when designing the microbiopsy device. These forces, in combination with beam bending calculations, eq. 1, allowed estimating minimum values for the prong length and thickness of the microbiopsy device, to safely avoid breakage during tissue penetration.

$${F}_{break} = \frac{{σ}_{break} I}{Lc} \qquad ( 1 )$$

where I denotes the bending moment of inertia, L the length of the prong and c the distance from the neutral axis of the beam. The breaking stress was estimated as the yield stress for <100> single crystal silicon (σ_{break}=7 GPa).

The microbiopsy devices were fabricated on an SOI wafer with a 90 μm device layer, utilizing Deep Reactive Ion Etching (DRIE); furnace oxidation to achieve sharp features, and; HF etching to free-etch the microbiopsy devices.

More specifically, the method comprises the steps of:

providing a silicon-on-insulator (SOI) wafer, wherein a thickness of the silicon layer is smaller than 1 mm;

depositing an oxide layer pattern on the SOI wafer, the pattern corresponding to a shape of the microbiopsy device;

subjecting the patterned SOI wafer to deep reactive-ion etching (DRIE) to remove the silicon surrounding the oxide pattern;

subjecting the DRIE-etched SOI wafer to furnace oxidation;

subjecting the furnace oxidised SOI wafer to HF etching to release the microbiopsy device from the SOI wafer.

The microbiopsy devices were mounted on Nitinol wires with a laser-cut front end matched to the back-end shape of the microbiopsy device (FIG. 5 (9)). The Nitinol wires were aligned above the microbiopsy device, at wafer-level, and vertically pushed onto the structure, creating a mechanical interlock. Breakaway structures with clearly defined crack initiation points temporarily supported the microbiopsy devices, making it easy to pull out the microbiopsy device from the wafer, once assembled on the wire. Finally, cyanoacrylate glue was applied for final fixation of the microbiopsy device to the wire.

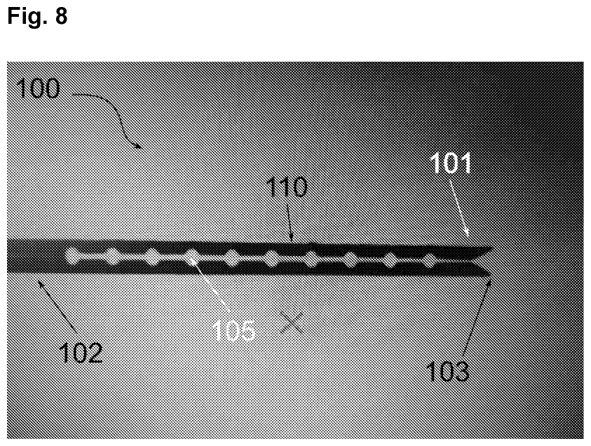

Referring now to FIG. 8, there is shown another embodiment of a microbiopsy device according to the present disclosure. In this embodiment, the recess is machined directly on the distal end of a nitinol wire, thus making the proximal end of the main body of the microbiopsy device integrally formed therewith. Similar to the embodiments of FIGS. 4a, 4b and 6a, 6b with the prongs, the recess comprises an opening in the transverse direction of the main body which is throughgoing. Instead of barbs, the embodiment of FIG. 8 comprises a longitudinal recess in the general form of a slit, with a plurality of circular notches regularly distributed along the length of the main body. The notches have a diameter larger than the width of the slit.

Ex-vivo tissue sampling was evaluated in a test rig where the microbiopsy devices were mounted above a liver sample. The microbiopsy devices were inserted and extracted from the sample in a controlled manner using a stepper motor and fixtures enabling independent motion of the Extroducer and microbiopsy device.

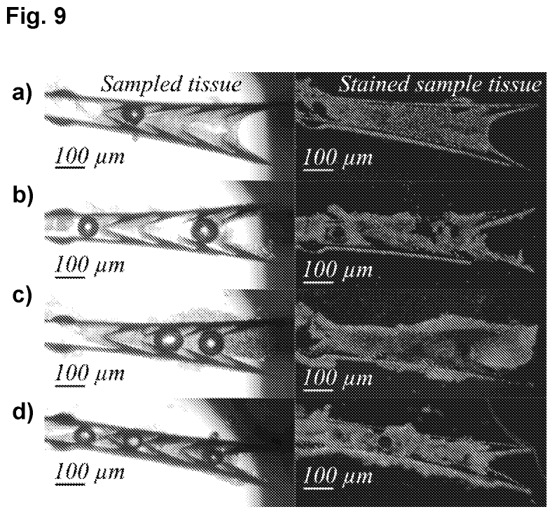

Samples from ex-vivo liver obtained in the test rig were stained with 4′,6-Diamidino-2-Phenylindole (DAPI), revealing the presence of cellular material, as seen in FIGS. 9a to 9d. On the left of the figures are brightfield images, and corresponding fluorescence images are on the right. The staining is performed directly after sampling by mounting the microbiopsy device on a glass slide, applying a mixture of DAPI and glycerol on top of the device and putting a cover slide on top. Cell nuclei counting in the images in FIG. 9 allowed estimating 500-1000 cells in each sampling.

A preferred embodiment of a microbiopsy device for collecting particles according to the invention has been described. However, the person skilled in the art realizes that this can be varied within the scope of the appended claims without departing from the inventive idea.

All the described alternative embodiments above or parts of an embodiment can be freely combined without departing from the inventive idea as long as the combination is not contradictory.

### LIST OF REFERENCE SIGNS

100 main body
101 distal end
102 proximal end
103 distal tip
104 mounting interface
105 recess
106 barb
107 longitudinal slit
108 transverse opening
109 cavity
110 flexible prong
111 bumper structure

1. A microbiopsy device for extracting a tissue sample, the microbiopsy device comprising a main body extending between a distal end and a proximal end and adapted to have or assume a shape of substantially uniform transverse width along the length of the main body, wherein
the distal end is arranged to enter tissue;
the proximal end comprises a mounting interface adapted for connection with an elongated member, or is integrally formed with a solid elongated member;
the transverse width of the main body is smaller than 1 mm; and
the main body comprises a recess extending in a longitudinal direction of the main body and defining a cavity arranged to capture tissue therein.

2. The microbiopsy device according to claim 1, wherein the transverse width of the main body is smaller than 500 μm, preferably smaller than 200 μm, most preferably smaller than 150 μm.

3. The microbiopsy device according to claim 1, wherein the volume of the cavity is smaller than 1 μl, preferably smaller than 250 nl, most preferably smaller than 100 nl.

4. The microbiopsy device according to claim 1, wherein the recess is machined in a distal end of the solid elongated member.

5. The microbiopsy device according to claim 1, wherein the recess comprises an opening in a transverse direction of the main body.

6. The microbiopsy device according to claim 1, wherein the recess extends towards the distal end of the main body, terminating in a distal opening.

7. The microbiopsy device according to claim 6, wherein the recess extends along substantially the whole length of the main body.

8. The microbiopsy device according to claim 1, wherein the main body has a substantially cylindrical shape.

9. The microbiopsy device according to claim 1, further comprising a plurality of barbs arranged within the recess and adapted to retain captured tissue.

10. The microbiopsy device according to claim 1, wherein the recess comprises a through-going opening to form two or more opposing prongs.

11. The microbiopsy device according to claim 10, wherein the prongs are flexible and adapted to be brought between a closed position, wherein the prongs are oriented in a longitudinal direction of the main body, and an open position, wherein the prongs expand beyond the transverse width of the main body.

12. The microbiopsy device according to claim 11, wherein the prongs comprise a bumper structure arranged on an outer surface.

13. The microbiopsy device according to claim 10, wherein the prongs have a thickness smaller than 500 μm.

14. The microbiopsy device according to claim 1, being manufactured in a material selected from a ceramic, a semiconductor, a metal or a polymer, or a combination thereof.

15. The microbiopsy device according to claim 1, being manufactured through additive manufacturing.

16. A biopsy device comprising:
an endoluminal access device having a flexible elongated hollow body terminating in a distal penetration portion arranged to penetrate a vascular tissue wall, wherein an outer diameter of the endoluminal access device is smaller than 1.2 mm;
an elongated member movably arranged inside the endoluminal access device and having an outer diameter corresponding to an inner diameter of the endoluminal access device; and
a microbiopsy device according to claim 1 mounted on or integrally formed with a distal end of the elongated member and having a transverse width substantially equal to the outer diameter of the elongated member.

17. The biopsy device according to claim 16, wherein the elongated member is made from a shape-memory alloy, preferably nitinol.

18. A method of extracting a tissue sample from a subject comprising:
providing a biopsy device comprising:
an endoluminal access device having a flexible elongated hollow body terminating in a distal penetration portion arranged to penetrate a vascular tissue wall, wherein an outer diameter of the endoluminal access device is smaller than 1.2 mm;
an elongated member movably arranged inside the endoluminal access device and having an outer diameter corresponding to an inner diameter of the endoluminal access device; and
a microbiopsy device comprising a main body extending between a distal end and a proximal end and adapted to have or assume a shape of substantially uniform transverse width along the length of the main body, wherein:
the distal end is arranged to enter tissue;
the proximal end comprises a mounting interface adapted for connection with an elongated member, or is integrally formed with a solid elongated member;
the transverse width of the main body is smaller than 1 mm; and
the main body comprises a recess extending in a longitudinal direction of the main body and defining a cavity arranged to capture tissue therein;
wherein the microbiopsy device is mounted on or integrally formed with a distal end of the elongated member and having a transverse width substantially equal to the outer diameter of the elongated member;
introducing the biopsy device into the vasculature of the subject with the microbiopsy device and elongated member in a retracted position wherein the distal end of the main body does not protrude from the distal penetration portion of the endoluminal access device;
advancing the biopsy device through the vasculature until a targeted sampling site is reached;
penetrating the vascular tissue wall using the distal penetration portion of the endoluminal access device;
advancing the elongated member through the endoluminal access device such that the microbiopsy device exits from the distal penetration portion of the endoluminal access device and enters into tissue at the sampling site to capture a tissue sample;
retracting the elongated member into the endoluminal access device such that the microbiopsy device re-enters the distal penetration portion of the endoluminal access device with the captured tissue sample; and
retracting the biopsy device from the vasculature.

19. The method according to claim 18, wherein the step of advancing the biopsy device through the vasculature comprises navigating inside the vasculature using x-ray guidance.
